(19) United States
(12) Patent Application Publication
(10) Pub. No.: US 2017/0224711 A1
(43) Pub. Date: Aug. 10, 2017

(54) COMPOSITIONS AND METHODS FOR THE TREATMENT OF NEURODEGENERATIVE DISEASES

(71) Applicant: Rathindra N. Bose, Houston, TX (US)

(72) Inventor: Rathindra N. Bose, Houston, TX (US)

(73) Assignee: University of Houston System, Houston, TX (US)

(21) Appl. No.: 15/497,511

(22) Filed: Apr. 26, 2017

Related U.S. Application Data
(62) Division of application No. 14/626,086, filed on Feb. 19, 2015.
(60) Provisional application No. 61/941,622, filed on Feb. 19, 2014.

(51) Int. Cl. A61K 31/688

(52) U.S. Cl. CPC A61K 31/688

(57) ABSTRACT

In some embodiments, the present disclosure pertains to a composition for modulating the expression of at least one gene associated with neuronal cell survival or stability. In some embodiments, the present disclosure provides for compositions for the treatment of neurodegenerative diseases comprising one or more phosphate complexes of platinum of the formulas I, II, III and IV as set forth in FIG. 1. In some embodiments, the present disclosure relates to a method of treating a neurodegenerative disorder comprising administering therapeutically effective amounts of at least one of the aforementioned compositions described supra to a subject in need thereof, such that the compounds are effective in modulating the expression of a gene selected from the group consisting of NMDA-receptor, ATF, PPT2, HPD, EGR2, SLC7A11, FosB, SQSTM1, TMEM106B, RAB27A, STOX1, and SLC39A3.

### CROSS REFERENCE TO RELATED APPLICATIONS

This application is a divisional application of U.S. Nonprovisional application Ser. No. 14/626,086, filed on Feb. 19, 2015, which claims priority to U.S. Provisional Application No. 61/941,622, filed on Feb. 19, 2014. The entirety of the aforementioned applications is incorporated herein by reference.

### BACKGROUND

Neurodegenerative diseases affect an estimated 50 million Americans each year, exacting an incalculable personal toll and an annual economic cost of hundreds of billions of dollars in medical expenses and lost productivity. One of the main aspects in understanding the initiation and progression of a neurodegenerative disease is to elucidate mechanisms that underlie or predispose a particular neuron to selective vulnerability. A number of genes have been identified that play a role in neuronal cell survival and stabilization. There is a need in the art to develop compositions and methods for modulating the expression of these genes for effective treatment of neurodegenerative diseases and disorders.

### BRIEF SUMMARY

In an embodiment, the present disclosure pertains to a composition for modulating the expression of at least one gene associated with neuronal cell survival and stability comprising: one or more isolated platinum complexes of platinum (II) and (IV) having the general formulas as set forth in FIG. 1, wherein R¹ and R² represent monodentate neutral ligands, each independently selected from substituted or unsubstituted aliphatic or substituted or unsubstituted aromatic amines, or a single bidentate neutral ligand R3, replacing both R¹ and R², selected from substituted or unsubstituted aliphatic or aromatic diamines, with R¹ and R² coordinated to the platinum metal center, and wherein when one of R¹ and R² is NH₃, the other of R¹ and R² is not NH₃ for monodentate ligands; and wherein S is independently selected from hydroxide, acetic acid, butyric acid, and alpha-hydroxy acids or charged species thereof coordinated to the platinum metal center. In certain embodiments, R¹ and R² are selected from amine, methyl amine, ethyl amine, propyl amine, isopropyl amine, butyl amine, cyclohexane amine, aniline, pyridine, and substituted pyridine. In certain embodiments, R³ is selected from ethylene-diamine and cyclohexanediamine. In certain embodiments pharmaceutically acceptable salts of the compounds are claimed. In some embodiments, the composition further comprises at least one pharmaceutically acceptable carrier such as a carrier, diluent, adjuvant, or vehicle. In an embodiment, the composition is effective in modulating expression of at least one gene associated with neuronal cell survival and stability. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3.

In an embodiment, the present disclosure provides a composition for the treatment or prevention of neurodegenerative diseases comprising one or more isolated platinum complexes of platinum (II) and (IV) having the general formulas as set forth in FIG. 1, wherein R¹ and R² represent monodentate neutral ligands, each independently selected from substituted or unsubstituted aliphatic or substituted or unsubstituted aromatic amines, or a single bidentate neutral ligand R3, replacing both R¹ and R², selected from substituted or unsubstituted aliphatic or aromatic diamines, with R¹ and R² coordinated to the platinum metal center, and where when one of R¹ and R² is NH₃, the other of R¹ and R² is not NH₃ for monodentate ligands; and where S is independently selected from hydroxide, acetic acid, butyric acid, and alpha-hydroxy acids or charged species thereof coordinated to the platinum metal center. In certain embodiments, R¹ and R² are selected from amine, methyl amine, ethyl amine, propyl amine, isopropyl amine, butyl amine, cyclohexane amine, aniline, pyridine, and substituted pyridine. In certain embodiments, R³ is selected from ethylene-diamine and cyclohexanediamine. In certain embodiments pharmaceutically acceptable salts of the compounds are claimed. In some embodiments of the present disclosure, the composition is effective in modulating the expression of at least one gene associated with neuronal cell survival and/or stability. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3. Specifically, the composition may be effective in treating neurodegenerative diseases selected from amyotropic lateral sclerosis, Alzheimer's disease, stroke, epilepsy, Parkinson's, Huntington's disease, and diabetes associated peripheral neuropathy. In some embodiments, the composition further comprises a therapeutically effective amount of one or more of the provided complexes and at least one pharmaceutically acceptable carrier such as a carrier, diluent, adjuvant, or vehicle. In an embodiment, the composition is effective in modulating expression of at least one gene involved in neurodegenerative disease or disorder. In an embodiment, the aforementioned composition is used in combination with standard therapy used for treating or preventing the neurodegenerative disease

Further embodiments of the present disclosure pertain to a method of treating or preventing neurodegenerative diseases in a subject in need thereof. Such a method comprises administering to the subject a therapeutically effective amount of at least one of the compositions described above. In a preferred embodiment, the subject is a mammal, such as a human, e.g., a subject diagnosed as having, or at risk for developing, a neurodegenerative disease. In an embodiment, the composition is effective in modulating expression of at least one gene involved in neurodegenerative disease or disorder. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3. Specifically, the composition may be effective in treating neurodegenerative diseases selected from amyotropic lateral sclerosis, Alzheimer's disease, stroke, epilepsy, Parkinson's, Huntington's disease, and diabetes associated peripheral neuropathy. In some embodiments the method further comprises administering at least one pharmaceutically acceptable carrier, diluent, adjuvant or a vehicle.

The treatment method of the present invention may also be combined with any other conventional treatment or treatment regime against a neurodegenerative disorder, and thus, the method in one embodiment further comprises administering at least one additional therapeutic agent specific for the neurodegenerative disease being treated.

Other embodiments of the present disclosure are directed towards a method for modulating the expression of at least one gene involved in neuronal cell survival and/or stability. Such a method comprises contacting the neuronal cell with an effective amount of at least one of the compositions described above. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3.

As set forth in more detail below, the compositions and methods of the present disclosure provide for treatment of neurodegenerative diseases via modulation of genes involved in neuronal cell survival.

### BRIEF DESCRIPTION OF THE FIGURES

FIG. 1 displays the general formulas for the isolated platinum(II) and (IV) compounds of the present invention.

FIGS. 2A-2F display structures of representative platinum (II) and platinum (IV) complexes of the present invention, namely diammine (dihydrogen pyrophospahto)platinum(II) (FIG. 2A), also known as am-2; cis-diammine-trans-dihydroxo(dihydrogen pyrophosphate platinum (IV) (FIG. 2B), also known as am-4; 1,2-ethanediamine(dihydrogen pyrophospahto)platinum(II) (FIG. 2C), also known as en-2; 1,2-ethanediamine-trans-dihydroxo(dihydrogen pyrophospahto) platinum(IV) (FIG. 2D), also known as EN-4; trans-1,2-cyclohexanediamine(dihydrogen pyrophospahto) platinum(II) (FIG. 2E), also known as dach-2; and trans-1,2-cyclohexanediamine)-trans-dihydroxo(dihydrogen pyrophospahto) platinum (IV) (FIG. 2F), also known as dach-4.

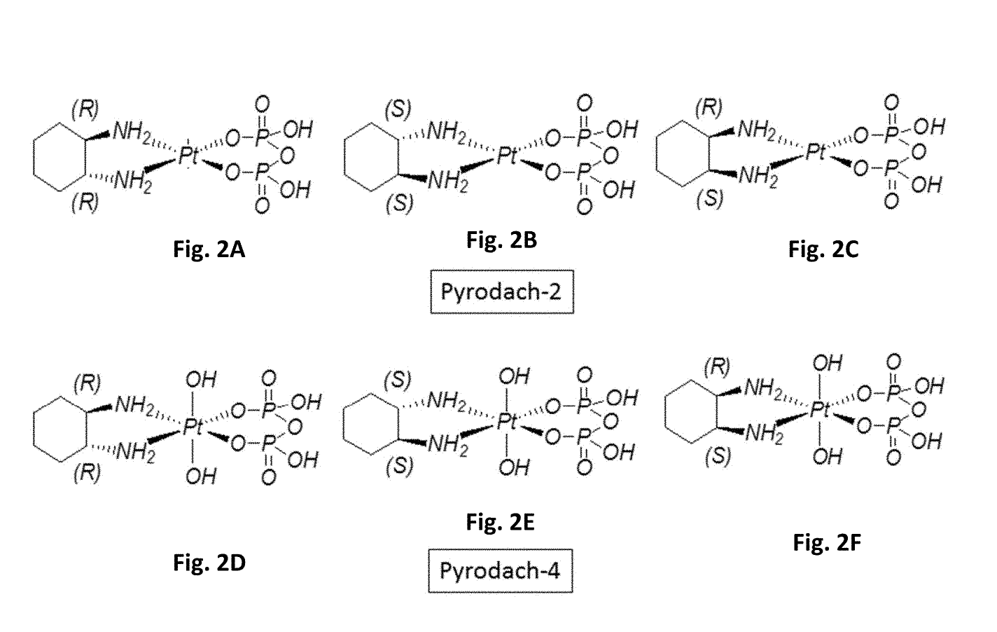

FIG. 3 shows a heat map reflecting the fold change in expressions of genes modulated by RRD2 involved in neuronal cell survival and stability. The scale was limited from −4 to +8. Genes expressed more than 8-fold was limited to +8. Note that the overexpressed genes are involved in preventing neurodegenerative diseases while the suppressed genes help in stopping the progression of the same.

FIGS. 4A-4C show modulation of protein expression of some of the representative genes associated with neuronal cell survival and stability listed in Table 1 by RRD2 (R,R-enantiomer of compound represented in FIG. 2A) and RRD4 (R,R-enantiomer of compound represented in FIG. 2D)(FIGS. 4A-4B). GAPDH and Actin were used as controls (FIG. 4C).

FIG. 5 shows Action potential (AP) threshold in RRD4 treated cells. RRD4 raised action potential (AP) threshold (N=6, P: 0.0455, unpaired t-test with Welch's correction). Membrane in RRD4 treated cells was significantly more depolarized. RRD4 pretreatment increased action potential threshold. Action potential threshold was measured at the beginning of the sharp upward rise of the depolarizing phase of the action potential. (*P<0.05).

FIG. 6 shows Action potential (AP) amplitude in RRD4 treated cells. AP amplitude decreased significantly in the presence of RRD4 (N=6, P: 0.0499, unpaired t-test with Welch's correction). RRD4 had no effect on input resistance (N=6, P: 0.8, unpaired t-test with Welch's correction). Pretreated slices with RRD4 had a lower peak potential amplitude. Mean values for the AP amplitude is voltage difference in 10-90% rise time in each action potential. (*P<0.05). Overall, RRD4 had a significant modulatory effect on neuronal action potential threshold and amplitude, but not the input resistance of neurons.

FIG. 7 shows RRD4 effect on seizure formation threshold. Time to seizure onset was significantly delayed by RRD4 (N=6, P: 0.0155, unpaired t-test with Welch's correction. RRD4 pretreatment prolonged the seizure onset time in seizure induced rats. Mean values for the seizure onset time as to latencies to seizures of each group. (*P<0.05).

### DETAILED DESCRIPTION

It is to be understood that both the foregoing general description and the following detailed description are exemplary and explanatory only, and are not restrictive of the invention, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims.

The disclosures of all patents, patent applications and publications cited herein are hereby incorporated herein by reference, to the extent that they provide procedural or other details consistent with and supplementary to those set forth herein.

A “neurodegenerative disease”, “neurological disease” or “neurological disorder” is any disease or disorder that affects the nervous system (the central or peripheral nervous system). Exemplary neurological diseases and disorders include Huntington's Disease (HD), Parkinson's Disease (PD), Amyotropic Lateral Sclerosis (ALS), Alzheimer's Disease, Lewy body dementia, Multiple System Atrophy, spinal and bulbar muscular atrophy (Kennedy's disease), Tourette Syndrome, Autosomal dominant spinocerebellar ataxia (SCA) (e.g., Type 1 SCA1, Type 2 SCA2, Type 3 (Machado-Joseph disease) SCA3/MJD, Type 6 SCA6, Type 7 SCAT, Type 8 SCAB, Friedreich's Ataxia and Dentatorubral pallidoluysian atrophy DRPLA/Haw-River syndrome), schizophrenia, age associated memory impairment, autism, attention-deficit disorder, bipolar disorder, and depression. As used herein, a “neurodegenerative disease” or “neurological disorders” or “neurological diseases” also refers to a disease in which degeneration occurs of either gray or white matter, or both, of the nervous system. Thus, such a disease can be diabetic neuropathy, senile dementias, Alzheimer's disease, Mild Cognitive Impairment (MCI), dementia, Lewy Body Dementia, Frontal Temporal Lobe dementia, Parkinson's Disease, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, prion diseases such as Creutzfeldt-Jakob disease, carpal tunnel syndrome, peripheral neuropathies associated with various diseases, including but not limited to, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, complications of various drugs (e.g., metronidazole) and toxins (e.g., alcohol or organophosphates), Charcot-Marie-Tooth disease, ataxia telangectasia, Friedreich's ataxia, amyloid polyneuropathies, adrenomyeloneuropathy, Giant axonal neuropathy, Refsum's disease, Fabry's disease and lipoproteinemia.

A “neuronal gene” is a gene expressed in neuronal cells. A neuronal gene can be expressed exclusively in neuronal cells, or can be expressed in other cell types in addition to the neuronal cell.

A “neuronal cell” is a cell of the nervous system, e.g., the peripheral or the central nervous system. A neuronal cell can be a nerve cell (i.e., a neuron), e.g., a sensory neuron or a motoneuron, or a glial cell. Exemplary neurons include dorsal root ganglia of the spinal cord, spinal motor neurons, retinal bipolar cells, cortical and striatal cells of the brain, hippocampal pyramidal cells, and purkinje cells of the cerebellum. Exemplary glial cells include oligodendrocytes and astrocytes of the central nervous system, and the Schwann cells of the peripheral nervous system.

As used herein, the term “modulate” or “modulating” refers to any change in expression of a gene of interest, including an increase or decrease in expression of a gene-of-interest. As such, modulation of a gene-of-interest can include over-expressing, under-expressing, or substantially blocking expression of the gene-of-interest in a cell.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide. Genes can be obtained from a variety of sources, including cloning from a source of interest or synthesizing from known or predicted sequence information, and can include sequences designed to have desired parameters. Examples of genes that can be modulated in accordance with the presently-disclosed subject matter include, but are not limited to, the following: NMDA-receptor, ATF, PPT2, HPD, EGR2, SLC7A11, FosB, SQSTM1, TMEM106B, RAB27A, STOX1, SLC39A3.

As used herein “therapeutically effective amount” is an art-recognized term. In certain embodiments, the term refers to an amount of a salt or composition disclosed herein that produces some desired effect at a reasonable benefit/risk ratio applicable to any medical treatment. In certain embodiments, the term refers to that amount necessary or sufficient to eliminate or reduce medical symptoms for a period of time. The effective amount may vary depending on such factors as the disease or condition being treated, the particular targeted constructs being administered, the size of the subject, or the severity of the disease or condition. One of ordinary skill in the art may empirically determine the effective amount of a particular composition without necessitating undue experimentation.

As used herein “subject” or “individual” or “patient,” may mean either a human or non-human animal, such as primates, mammals, and vertebrates.

The compounds described herein are useful for the prevention and/or treatment of neurodegenerative diseases or conditions including Alzheimer's disease, Parkinson's disease, motor neuron diseases such as amyotrophic lateral sclerosis, and other neurodegenerative diseases.

The compounds described herein are useful for modulating expression of genes involved in neuroprotection or neuronal cell survival and stability.

### Phosphaplatins

Phosphaplatins are phosphate bound platinum(II) and platinum(IV) coordination compounds and are described in U.S. Pat. Nos. 7,700,649 and 8,034,964 and U.S. patent application Ser. No. 13/701,313 and fully incorporated herein by reference. Methods of synthesizing and isolating stable platinum (II) and (IV) pyrophosphate complexes are also described in U.S. Pat. Nos. 770,649, 8,034,694, and U.S. patent application Ser. No. 13/701,313 and fully incorporated herein by reference. The pyrophosphate coordinated platinum-(II) and -(IV) compounds disclosed herein show excellent antitumor activities against a variety of human cancers as demonstrated by both in vitro (Bose et al., 2008) and in vivo experiments using Scid and Nude mice (Bose et al., 2012). Moreover, these compounds show reduced toxicity compared to other platinum chemotherapeutics that are currently being used as cancer chemotherapies. Usefulness of various Phosphaplatins, as effective anticancer agents, their potential in reducing neurotoxicity associated with chemotherapeutic drugs, or as anti-angiogenic agents has been reported in literature.

One of the main aspects in understanding the initiation and progression of a neurodegenerative disease is to elucidate mechanisms that underlie or predispose a particular neuron to selective vulnerability. The present disclosure is directed towards the use of phosphaplatins to modulate expression of genes involved in neuronal cell survival and stability, for example NMDA-receptor, ATF, PPT2, HPD, EGR2, SLC7A11, FosB, SQSTM1, TMEM106B, RAB27A, STOX1, and SLC39A3.

### NMDAR2C (GRIN2C)

N-methyl-D-aspartate (NMDA) receptors are a class of ionotropic glutamate receptors involved in a number of important neuronal activities in mammalian nervous systems including neuronal migration, synaptogenesis, neuronal plasticity, neuronal survival, and excitotoxicity. These receptor channels are heteromers composed of the key receptor subunit NMDAR1 (GRIN1) and 1 or more of the 4 NMDAR2 subunits: NMDAR2A (GRIN2A), NMDAR2B (GRIN2B), NMDAR2C (GRIN2C), and NMDAR2D (GRIN2D). NMDA channel has been shown to be involved in an activity-dependent increase in the efficiency of synaptic transmission thought to underlie certain kinds of memory and learning. N-methyl D-aspartate receptor 3, epsilon 3 subunit glutamate receptor, is expressed in various tissues like the brain, mainly in cerebellum, basal ganglia, in the heart, skeletal muscle, and the pancreas. The decreased expression of this protein has been implicated with pathophysiological conditions such as Parkinson's disease, Alzheimer's disease, depression, and schizophrenia. Stabilization or upregulation of this gene and associated protein is associated with neuronal cell survival and stability and hence has been shown to impart a neuroprotective effect in cells.

### ATF3 (Activating Transcription Factor 3)

Activating transcription factor 3 (ATF3) belongs to the ATF/cyclic AMP responsive element binding family of transcription factors. It has been variously described as an immediate early gene, a stress inducible gene and an adaptive response gene. In neurons, ATF3 expression is closely linked to neuronal survival and the regeneration of axons following axotomy. The levels of ATF3 mRNA and protein are normally very low in neurons and glia but their expression is rapidly upregulated in response to injury. ATF3 expression is modulated mainly at the transcriptional level and has markedly different effects in different types of cell. ALS is a fatal neurodegenerative disease characterized by loss of motor function. In an ALS mouse model, ATF3 overexpression in motor neurons was shown to result in a modified gene expression driving the neurons into a pro-survival and pro-regenerative state, increasing motor neuron survival and maintaining axonal connection with muscle by promoting axonal sprouting.

### PPT2 (Palmitoyl-Protein Thioesterase 2)

The PPT2 gene encodes a member of the palmitoyl-protein thioesterase family. Palmitoyl protein thioesterases are lysosomal hydrolases that remove thioester-linked fatty acyl groups such as palmitate from modified cysteine residues in proteins or peptides during lysosomal degradation. Deficiency of PPT2 in humans has been linked with infantile neuronal ceroid lipofuscinosis (infantile Batten disease), a neurodegenerative disorder.

### HPPD (4-Hydroxyphenylpyruvate Dioxygenase)

The protein encoded by the HPPD gene is an Fe(II)-containing non-heme oxygenase that catalyzes the second reaction in the catabolism of tyrosine—the conversion of 4-hydroxyphenylpyruvate into homogentisate, that is common to essentially all aerobic forms of life. Tyrosinemia type 3 (TYRO3) and hawkinsinuria (HAWK) are caused by defects in this gene.

### EGR2 (Early Growth Response 2)

The Early Growth response protein 2 is a zinc finger transcription factor. Defects in this gene are associated with Charcot-Marie-Tooth disease type 1D (CMT1D), Charcot-Marie-Tooth disease type 4E (CMT4E), a disease characterized by congenital hypomyelination neuropathy, and with Dejerine-Sottas syndrome (DSS).

### ASTN1 and ASTN2

This protein is encoded by the Astrotactin 1 gene and may function in neuronal migration. Specifically, ASTN1 functions as a neuronal-glial ligand during CNS glial-guided migration. Individuals with schizophrenia show a deletion at this locus. During glial-guided migration at different developmental stages, another family member, ASTN2 is expressed at high levels in migrating cerebellar granule neurons, along with ASTN1. Studies indicate that ASTN2 regulates the levels of ASTN1 in the plasma membrane and that the release of neuronal adhesion to the glial fiber during neuronal locomotion involves the intracellular trafficking of ASTN1.

### SLC7A11 (Solute Carrier Family 7 (Anionic Amino Acid Transporter Light Chain, Xc-System), Member 11)

SLC7A11 (or xCT), together with SLC3A2 (or 4F2hc), encodes the heterodimeric amino acid transport system x_{c}⁻, which mediates entry of cystine into the cell coupling to efflux of glutamate. SLC7A11 has been identified as the predominant mediator of Kaposi sarcoma-associated herpes virus fusion and entry permissiveness into cells. Increased expression of this gene in primary gliomas (compared to normal brain tissue) was associated with increased glutamate secretion via the XCT channels, resulting in neuronal cell death

### FosB (FBJ Murine Osteosarcoma Viral Oncogene Homolog B)

The FosB gene encodes a leucine zipper protein that dimerizes with the Jun family of proteins to form a transcription factor complex, AP-1. As such, the FOS proteins have been implicated as regulators of cell proliferation, differentiation, and transformation. FosB-null mice display impaired adult hippocampal neurogenesis and spontaneous epilepsy with depressive behavior.

### SQSTM1 (Sequestosome 1)

This gene encodes a ubiquitin-binding multifunctional protein that regulates activation of the nuclear factor kappa-B (NF-kB) signaling pathway. This gene may also be involved in cell differentiation, apoptosis, immune response and regulation of K (+) channels. Mutations in this gene result in sporadic and familial Paget disease of bone. Additionally, SQSTM1 gene mutations could be the cause or genetic susceptibility factor of ALS in some patients.

### TMEM106B (Transmembrane Protein 106B)

TMEM106B gene encodes a transmembrane protein. TMEM106B genotype, characterized by 3 particular single-nucleotide polymorphisms, is strongly correlated with frontotemporal lobar degeneration with TAR DNA-binding protein (TDP-43) inclusions (FTLD-TDP). The most significant association of TMEM106B single nucleotide polymorphisms with risk of FTLD-TDP was observed in patients with progranulin (GRN) mutations. Front-temporal lobar degeneration (FTLD) is the second most common cause of pre-senile dementia, an incurable neurodegenerative disorder. Studies indicate that decreasing TMEM106B levels might result in neuronal cell survival and stability and hence protective effects.

### RAB27A (Ras-Related Protein Rab-27A)

Upregulation of this protein in basal forebrain neurons has been associated with mild cognitive impairment and Alzheimer's disease. The RAB27A gene encodes a membrane-bound protein belonging to the GTPase superfamily and may be involved in protein transport and small GTPase mediated signal transduction. Mutations in this gene are associated with Griscelli syndrome type 2.

### STOX1(Storkhead-Box Protein 1)

STOX1 is a transcription factor which is functionally and structurally homologous to the forkhead box protein family. Diseases associated with STOX1 include eclampsia, and pre-eclampsia/eclampsia (PEE4). Intraneuronal fibrillary tangles are a major hallmark of several neurodegenerative diseases including Alzheimer's disease. STOX1A induces phosphorylation of the longest human tau isoform at phospho-epitopes typically found in neurofibrillary tangles in Alzheimer's disease.

### RhoGAP2

This gene encodes a member of the GTPase activating protein family which activates a GTPase belonging to the RAS superfamily of small GTP-binding proteins. Rho family of GTPases and related molecules play an important role in various aspects of neuronal development, including neurite outgrowth and differentiation, axon path finding, and dendritic spine formation and maintenance.

### SLC39A3 (Solute Carrier Family 39 (Zinc Transporter), Member 3)

SLC39A3 is a zinc transporter that apparently plays a critical role in zinc homeostasis. Knockout of Zn transporters Zip-1 and Zip-3 attenuates seizure-induced CA1 neurodegeneration.

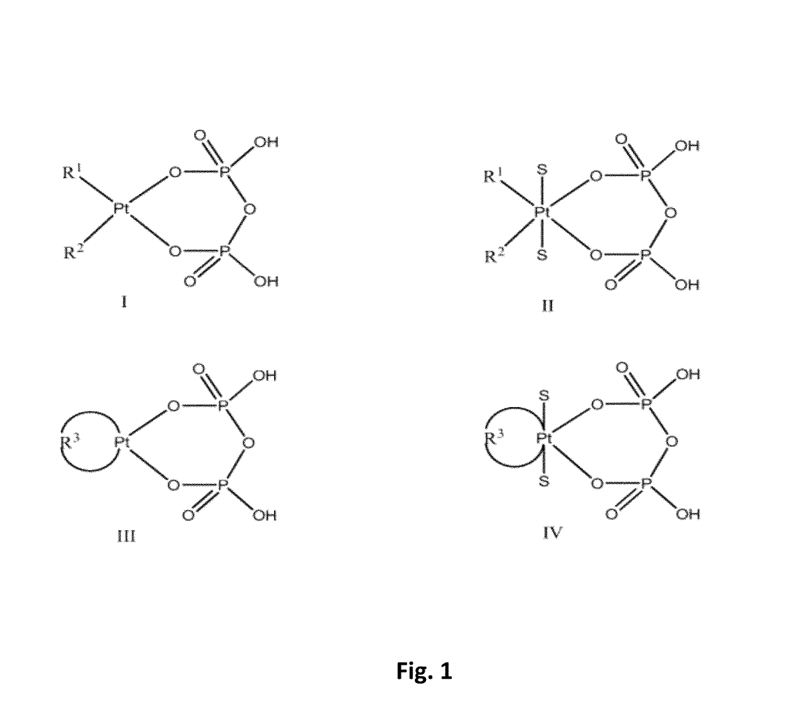

Accordingly, in an embodiment, the present disclosure relates to a composition for modulating the expression of at least one gene associated with neuronal cell survival and stability. In an embodiment, such a composition comprises one or more isolated platinum complexes of platinum (II) and (IV) having the general formulas as set forth in FIG. 1, wherein R¹ and R² represent monodentate neutral ligands, each independently selected from substituted or unsubstituted aliphatic or substituted or unsubstituted aromatic amines, or a single bidentate neutral ligand replacing both R1 and R2 selected from substituted or unsubstituted aliphatic or aromatic diamines, with R1 and R2 coordinated to the platinum metal center, and wherein when one of R¹ and R² is NH₃, the other of R¹ and R² is not NH₃ for monodentate ligands; and wherein S is independently selected from hydroxide, acelic acid, butyric acid, and alpha-hydroxy acids or charged species thereof coordinated to the platinum metal center. In certain embodiments, R¹ and R² are selected from amine, methyl amine, ethyl amine, propyl amine, isopropyl amine, butyl amine, cyclohexane amine, aniline, pyridine, and substituted pyridine. In certain embodiments, R³ is selected from ethylene-diamine and cyclohexanediamine. In certain embodiments, pharmaceutically acceptable salts of the compounds are claimed. In some embodiments, the composition further comprises a therapeutically effective amount of one or more of the provided complexes and at least one pharmaceutically acceptable carrier such as a carrier, diluent, adjuvant, or vehicle. In an embodiment, the isolated platinum complex is 1,2-ethanediamine(dihydrogen pyrophosphato) platinum (II). In another embodiment the isolated monomeric platinum complex is (Trans-1,2-cyclohexanediamine)(dihydrogen pyrophosphato) platinum(II). In yet another embodiment, the isolated monomeric platinum complex is cis-diammine-trans-dihydroxo(dihydrogen pyrophosphato)platinum(IV). In yet, still another embodiment, the isolated monomeric platinum complex is 1,2-Ethanediamine-trans-dihydroxo(dihydrogen pyrophosphato)platinum(IV). In some embodiments, the isolated monomeric complex is Trans-1,2-cyclohexanediamine)-trans-dihyroxo(dihydrogen pyrophosphato)platinum(IV). In an embodiment, the composition may be effective in treating neurodegenerative diseases selected from amyotropic lateral sclerosis, Alzheimer's disease, stroke, epilepsy, Parkinson's, Huntington's disease, diabetes associated peripheral neuropathy leg and foot ulcerations associated with diabetes, pain and sleep loss induced by diabetes associated neuropathy. In some embodiments, the composition is effective in modulating the action potential in the neuronal cell. In some embodiments the at least one gene is selected from the group consisting of but not limited to NMDA-receptor, ATF, PPT2, HPD, EGR2, SLC7A11, FosB, SQSTM1, TMEM106B, RAB27A, STOX1, SLC39A3.

In an embodiment, the present disclosure pertains to a composition for the treatment and/or prevention of neurodegenerative diseases via modulation of at least one gene involved in neuronal cell survival and stability. In some embodiments the composition comprises one or more isolated platinum complexes of platinum (II) and (IV) having the general formulas as set forth in FIG. 1, wherein R¹ and R²represent monodentate neutral ligands, each independently selected from substituted or unsubstituted aliphatic or substituted or unsubstituted aromatic amines, or a single bidentate neutral ligand replacing both R1 and R2 selected from substituted or unsubstituted aliphatic or aromatic diamines, with R1 and R2 coordinated to the platinum metal center, and wherein when one of R¹ and R²is NH₃, the other of R¹ and R² is not NH₃ for monodentate ligands; and wherein S is independently selected from hydroxide, acetic acid, butyric acid, and alpha-hydroxy acids or charged species thereof coordinated to the platinum metal center. In certain embodiments, R¹ and R² are selected from amine, methyl amine, ethyl amine, propyl amine, isopropyl amine, butyl amine, cyclohexane amine, aniline, pyridine, and substituted pyridine. In certain embodiments, R³ is selected from ethylene-diamine and cyclohexanediamine. In certain embodiments, pharmaceutically acceptable salts of the compounds are claimed. In some embodiments, the composition further comprises a therapeutically effective amount of one or more of the provided complexes and at least one pharmaceutically acceptable carrier such as a carrier, diluent, adjuvant, or vehicle.

In an embodiment, the isolated platinum complex is 1,2-Ethanediamine(dihydrogen pyrophosphato) platinum (II). In another embodiment the isolated monomeric platinum complex is (Trans-1, 2-cyclohexanediamine) (dihydrogen pyrophosphato) platinum (II). In yet another embodiment, the isolated monomeric platinum complex is cis-diammine-trans-dihydroxo(dihydrogen pyrophosphato)platinum(IV). In yet, still another embodiment, the isolated monomeric platinum complex is 1,2-Ethanediamine-trans-dihydroxo(dihydrogen pyrophosphato)platinum(IV). In some embodiments, the isolated monomeric complex is Trans-1,2-cyclohexanediamine)-trans-dihyroxo(dihydrogen pyrophosphato)platinum(IV). In some embodiments the at least one gene is selected from the group consisting of but not limited to NMDA-receptor, ATF, PPT2, HPD, EGR2, SLC7A11, FosB, SQSTM1, TMEM106B, RAB27A, STOX1, SLC39A3.

In an embodiment, the composition may be effective in treating neurological diseases selected from amyotropic lateral sclerosis, Alzheimer's disease, stroke, epilepsy, Parkinson's, Huntington's disease, diabetes associated peripheral neuropathy leg and foot ulcerations associated with diabetes, pain and sleep loss induced by diabetes associated neuropathy.

Further embodiments of the present disclosure pertain to a method of treating and/or preventing neurodegenerative diseases in a subject in need thereof. Such a method comprises administering to the subject a therapeutically effective amount of at least one of the compositions described above. In a preferred embodiment, the subject is a mammal, such as a human, e.g., a subject diagnosed as having, or at risk for developing, a neurological disorder. In an embodiment, the composition is effective in modulating expression of at least one gene involved in neurodegenerative disease or disorder. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3. Specifically, the composition may be effective in treating neurodegenerative diseases selected from amyotropic lateral sclerosis, Alzheimer's disease, stroke, epilepsy, Parkinson's, Huntington's disease, and diabetes associated peripheral neuropathy. In some embodiments the method further comprises administering at least one pharmaceutically acceptable carrier, diluent, adjuvant or a vehicle.

The treatment method of the present invention may also be combined with any other conventional treatment or treatment regime against a neurodegenerative disorder, and thus, the method in one embodiment further comprises administering at least one additional therapeutic specific for the neurodegenrative disease being treated.

Other embodiments of the present disclosure are directed towards a method for modulating the expression of at least one gene involved in neuronal cell survival and stability. Such a method comprises contacting the neuronal cell with an effective amount of at least one of the compositions described above. In an embodiment, the composition is effective in modulating expression of at least one gene involved in imparting neuronal cell survival and/or stability. In some embodiments the gene is selected from the group consisting of but not limited to NMDA-receptor, ATF, PPT2, HPD, EGR2, SLC7A11, FosB, SQSTM1, TMEM106B, RAB27A, STOX1, SLC39A3.

In some embodiments the method further comprises administering at least one pharmaceutically acceptable carrier, diluent, adjuvant or a vehicle. The treatment method of the present invention may also be combined with any other conventional treatment or treatment regimens against a neurodegenerative disorder, and thus, the method in one embodiment further comprises administering at least one additional therapeutic agent or modality specific for the neurodegenerative disease being treated.

The methods of treatment disclosed herein are administered in accordance with good medical practice, taking into account the clinical condition of individual patient, the site and method of administration, scheduling of administration, sex, age, body weight and other factors of the patient. The therapeutically “effective amount”, for purposes of treatment herein, are thus determined by such considerations as are known in the art. The amount must be effective to achieve improvement, including but not limited to a more rapid recovery, or improvement or elimination of symptoms and other indicators may be selected as appropriate measures of therapeutically “effective amount” by those skilled in the art.

In the methods of treatment and/or prevention of the present disclosure, the complexes disclosed herein can be administered in various ways. It should be noted that they can be administered as the complex and can be administered alone in aqueous solution taking advantage of the excellent solubility of these complexes, or as an active ingredient in combination with pharmaceutically acceptable carriers, diluents, adjuvants and vehicles. The complexes can be administered orally, subcutaneously or parenterally including intravenous, intraarterial, intramuscular, intraperitoneally, intratonsillar, and intranasal administration as well as intrathecal and infusion techniques. Implants of the complexes are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The present disclosure also contemplates prodrugs of the compositions disclosed herein, as well as pharmaceutically acceptable salts of said prodrugs.

The doses can be single doses or multiple doses over a period of several days. The treatment generally has a length proportional to the length of the disease process and drug effectiveness and the patient species being treated.

The compositions disclosed herein may be administered to a subject in need of treatment by a variety of conventional routes of administration, including orally, topically, parenteral, e.g., intravenously, subcutaneously or intramedullary. Further, the compositions may be administered intranasally, as a rectal suppository, or using a “flash” formulation, i.e., allowing the medication to dissolve in the mouth without the need to use water. Furthermore, the compositions may be administered to a subject in need of treatment by controlled release dosage forms, site specific drug delivery, transdermal drug delivery, patch (active/passive) mediated drug delivery, by stereotactic injection, or in nanoparticles. When administering the complexes of the present disclosure parenterally, they will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propyleneglycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for the compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present disclosure, however, any vehicle, diluent, or additive used would have to be compatible with the complexes.

Sterile injectable solutions can be prepared by incorporating the complexes utilized in practicing the present invention in the required amount of the appropriate solvent with various other ingredients, as desired.

A pharmacological formulation of the present invention can be administered to the patient in an injectable formulation containing any compatible carrier, such as various vehicle, adjuvants, additives, and diluents; or the complexes utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Many other such implants, delivery systems, and modules are well known to those skilled in the art.

From the above disclosure, a person of ordinary skill in the art will recognize that the methods and systems of the present disclosure can have numerous additional embodiments. Reference will now be made to more specific embodiments of the present disclosure and experimental results that provide support for such embodiments. However, Applicants note that the disclosure below is for exemplary purposes only and is not intended to limit the scope of the claimed invention in any way.

### EXAMPLE 1

### CHIP Array: Whole Genome Expression

Human Ovarian cancer cells, A2780, were treated with compound and different concentration for 12 or 24 hours. The total RNA from the treated human cells were extracted by Trizol (Invitrogen) and purified by Rneasy Mini kit (Qiagen, Valencia, Calif.). The concentration and integrity of all RNA samples were assessed using the NanoDrop ND-2000 spectrophotometer (NanoDrop Technologies, Wilmington, Del.) and the Bioanalyzer 2100 system (Agilent Technologies, Santa Clara, Calif.). 1 ug of total RNA from each sample were subjected to whole-genome gene expression analysis at the Microarray Core Facility of University of Texas Southwestern Medical Center (https://microarray.swmed.edu) using the HumanHT-12 v4.0 BeadChip (Illumina, San Diego, Calif.) according to the manufacturer's instructions. Microarray data were extracted using BeadStudio v3.1 software, background-subtracted, and normalized using a cubic spline algorithm. Genes differentially expressed between groups were identified using the Illumina custom error model implemented in BeadStudio. Genes were considered significantly differentially expressed when P values were less than 0.05 and the change was greater than 2.0-fold. The pathways and interaction networks that the genes involved were further analyzed by Ingenuity Pathyway analysis software (www.ingenuity.com).

### EXAMPLE 2

### Neuromodulatory Actity of RRD4

Sprague-Dawley male rats (P14-18) were deeply anesthetized with ether and after the decapitation, the brains were removed rapidly and laced in the fresh high sucrose dissection solution. After hippocampus isolation, hippocampal slices (350 μM) were prepared using a Vibratome. The prepared slices were incubated in the artificial cerebrospinal fluid (ACSF) (pH 7.3) for half an hour. After incubating period, slices were transferred to recording chamber while oxygenated ACSF was continuously perfused constantly with the rate of 2 ml/min at 30° C. Borosilicate glass recording electrodes were filled with 0.9% NaCl (1-2 MΩ) and used for extracellular field potential recordings. Extracellular recordings were done from the border area between radiatum and pyramidale layers. Filled micropipettes (4-7 MΩ) containing intracellular solution were used for whole cell current-clamp recordings in the CA1 pyramidale layer. To elicit action potential activity, depolarizing square wave current pulses incremented by 20 pA were injected into the somas for 500-1000 ms, followed by 10 ms return to the baseline holding membrane potential (−80 mV). In addition, to square wave current pulses, slow depolarizing ramp-like current injections were used to quantify action potential threshold values. Neuronal membrane and field potential recordings were performed in control and in the RRD4 groups. RDD4 group brain slices were perfused with 20 μM RRD4. Zero magnesium model of inducing epileptiform activities was used in these experiments. In the treated group slices were incubated in 20 μM RRD4 for 15 minutes, and then were exposed to zero magnesium solution.

### EXAMPLE 3

### Western Blot Protocol

A2780 ovarian cancer cells cell line was cultured in 60 or 100 mm³ cell culture dishes. Once the plated were 80% confluent, the cells were incubated in the presence and absence of either R,R-D2 or R,R-D4. After 24 hrs of drug treatment, proteins were extracted from the cells using 200-500 μl of either RIPA or MPER protein extraction reagent buffer or both. Halt protease inhibitor cocktail from Thermo Fisher was added to the protein extraction reagents. The protein concentration was measured using BCA kit from Promega. The freshly extracted proteins (40 to 50 μg) were run on a 4-12% or 4-20% tris-glycine gels, at 120 V for 2 to 3 hours after which the proteins were transferred onto nitrocellulose membranes for one hour. Then the blots were incubated in 5% milk. The following primary antibodies were incubated overnight in 5% milk. These antibodies include: mouse anti-rabbit IgG-FITC, mouse anti-goat IgG-PE, anti-mouse IgG2a-PE, SQSTM1 (D-3), Egr-2 (H-220), ZIP3 (D-14), TOX1 (T-12), ATF-3 (H-90), NMDAμ4 (C-20), HPPD (H-300), Fos B (C-20), PPT2 (C-18), Hep G2 Cell Lysate, TMEM106B (C-12), xCT (Q-18) after which the proteins were detected by species-specific HRP-conjugated secondary antibody using ECL chemiluminescence kit purchased from GE health care.

### EXAMPLE 4

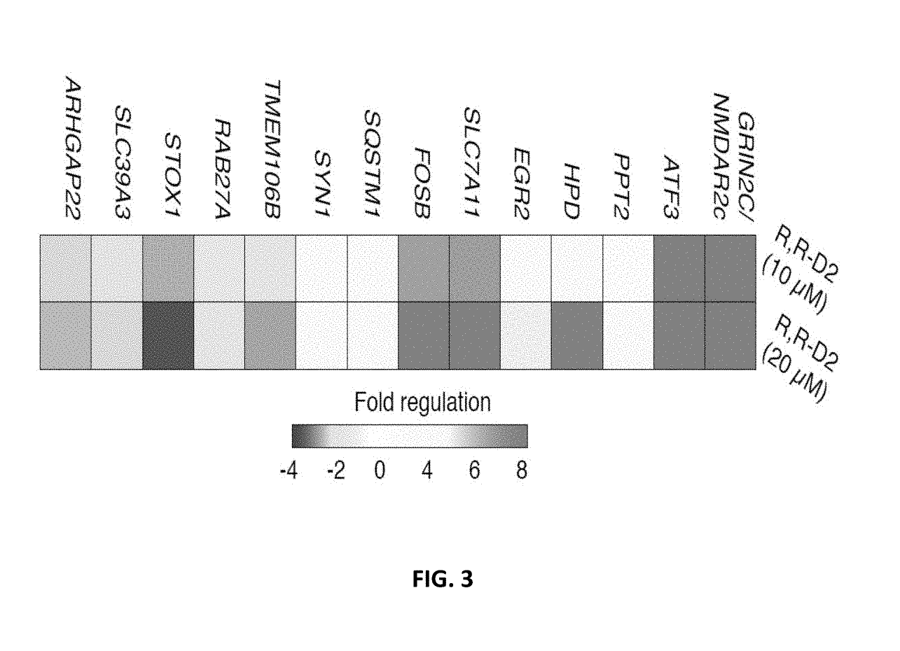

The treatment of A2780, human ovarian cancer cell line at different concentrations were examined for the differential gene expression compared to the whole human genome and compared to untreated samples (controls). A representative heat map reflecting the fold change in expressions of genes modulated by RRD2 involved in neurodegenerative diseases is shown in FIG. 3. Table 1 shows the modulation of genes following treatment with Phosphaplatins as compared to untreated samples. These genes are implicated in neurological disorders and neurodegenerative diseases and the modulation of these genes by Phosphaplatins is significant for providing neuroprotection.

Table 1 Fold change expression of specific genes implicated in imparting neuroprotective properties after treatment with RRD2 and RRD4 as compared to control or untreated cells.

TABLE 1

| Gene | Disease Implications | Change |
|---|---|---|
| NMDAR2C | NMDA-receptor-implicated with Alzheimer, Parkinson's, | 8↑ |
| | schizophrenia, depression | |
| ATF | Neurons survival and regeneration | 10↑ |
| PPT2 | PPT1and-2 deficiency in humans causes a neurodegenerative | 2-3↑ |
| | disorder, infantile neuronal ceroid lipofuscinosis (also known as | |
| | infantile Batten disease) | |
| HPD | Tyrosinemia type 3, caused by a genetic deficiency of 4- | 4-10↑ |
| | hydroxyphenylpyruvic acid dioxygenase (HPD) in tyrosine | |
| | catabolism, is characterized by convulsion, ataxia, and mental | |
| | retardation. | |
| EGR2 | Homozygous deletion of EGR2 enhances congenital | 2-4↑ |
| | amyelinating neuropathy. | |
| SLC7A11 | The overexpression of the cystine-glutamate exchanger, system | 6-8↑ |
| | Xc-, has been demonstrated as being neuroprotective | |
| FosB | fosB-null mice display impaired adult hippocampal | 4-23↑ |
| | neurogenesis and spontaneous epilepsy with depressive | |
| | behavior | |
| SQSTM1 | SQSTM1 gene mutations could be the cause or genetic | 2-6↑ |
| | susceptibility factor of ALS in some patients. | |
| TMEM106B | TMEM106B is a genetic risk factor for frontotemporal lobar | 2-3↓ |
| | degeneration. Amyotrophic lateral sclerosis (ALS), like FTLD- | |
| | TDP, is characterized by pathological TDP-43 inclusions. | |
| RAB27A | Upregulated expression of rab4, rab5, rab7, and rab27 | 1.7↓ |
| | correlated with antemortem measures of cognitive decline in | |
| | individuals with mild cognitive impairment (MCI) and AD. | |
| STOX1 | Intranueronal fibrillary tangles are a major hallmark of several | 2.5-3.6↓ |
| | neurodegenerative diseases including Alzheimer's disease. | |
| | STOX1A induces phosphorylation of the longest human tau | |
| | isoform at phosphor-epitopes typically found in neurofibrillary | |
| | tangles in Alzheimer's disease. | |
| SLC39A3 | Knockout of Zn transporters Zip-1 and Zip-3 attenuates seizure- | 2↓ |
| | induced CA1 neurodegeneration. | |

### EXAMPLE 5

### Protein Expression

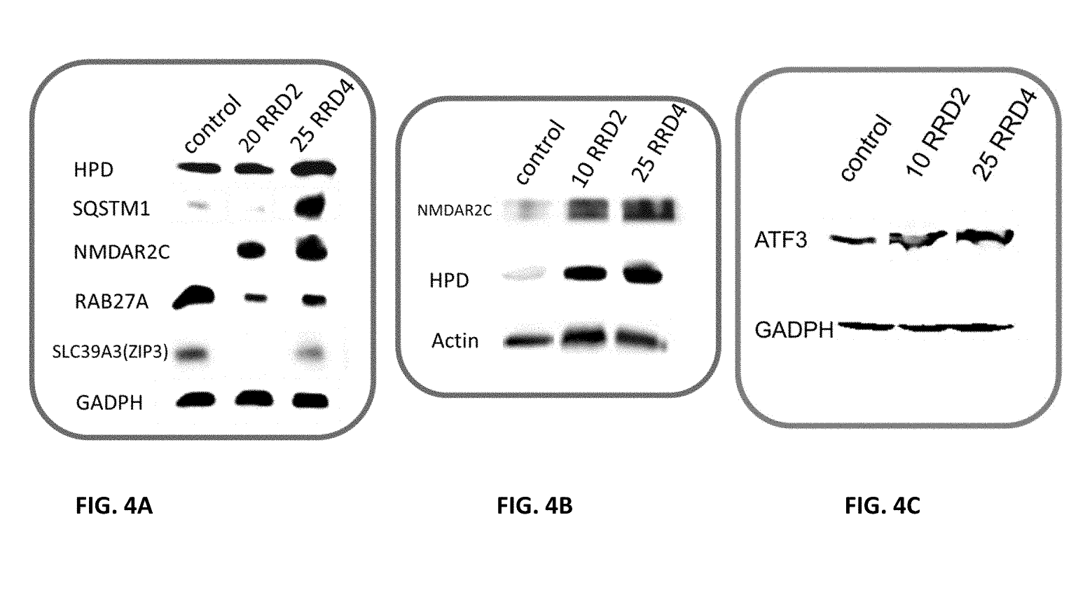

To confirm that the modulation of the genes listed in Table 1, following treatment with RRD2 and RRD4 in the human ovarian cancer cell line, A2780, also affects the protein expression of these genes, some representative genes from Table 1 were assessed for their corresponding protein expression as measured by Western Blot using protein specific antibodies. The protein expressions for HPD, SQSTM1, ATF3, NMDAR2C were higher compared to the control samples while RAB27A and SLC39A3 (ZIP3) expressions were lower than the control samples following treatment with the aforementioned compounds (FIGS. 4A-4B). Note that HPD and NMDAR2C were calibrated against both actin and GADPH (FIG. 4C). As indicated in the Tables, overexpressed proteins/genes are implicated in protecting neurons from a variety of neurodegenerative diseases. For example, SQSTM is barely expressed in untreated cells while cells treated with 25 μM RRD4 showed enhanced expression of protein (FIG. 4A). Likewise, down-regulated genes/proteins are connected with the onset or propagation of neurodegenerative diseases. For example, RAB27A is overexpressed in untreated cells as compared to cells treated with 20 μM RRD2 and 25 μM RRD4 (FIG. 4A). Hence, suppression of expressions the latter group of proteins by RRD2 and RRD4 is expected to prevent the progression of the diseases.

### EXAMPLE 6

### RRD4 Modulates Neural Action Potential Characteristics

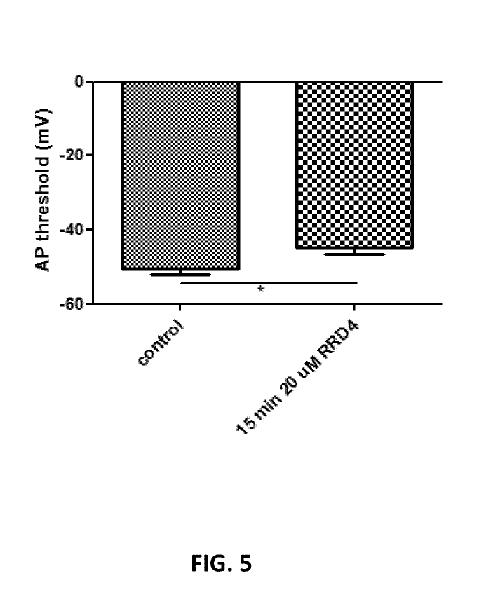

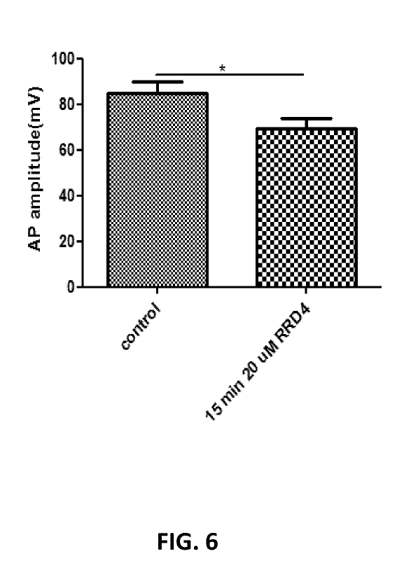

The neurodegeneration observed in many diseases has been associated with a progressive decrease in neuronal activity and synaptic dismantling is associated with many neurodegenerative diseases. A potential strategy for preventing and/or halting neurodegenerative disease is by modulating intrinsic properties of individual neurons. Applicants observed that RRD4 pretreatment increased action potential threshold (FIG. 5). Action potential threshold was measured at the beginning of the sharp upward rise of the depolarizing phase of the action potential. (*P<0.05). Based on the data, RRD4 increases action potential (AP) threshold and decreases its amplitude. The results suggest that RRD4 could modulate sodium or potassium channels and currents underlying the action potentials. Further, pretreated slices with RRD4 had lower peak potential amplitude (FIG. 6). Mean values for the AP amplitude is voltage difference in 10-90% rise time in each action potential. (*P<0.05). Overall, RRD4 had a significant modulatory effect on neuronal action potential threshold and amplitude, but not the input resistance of neurons.

### EXAMPLE 7

### RRD4 has Anti-Convulsant Properties

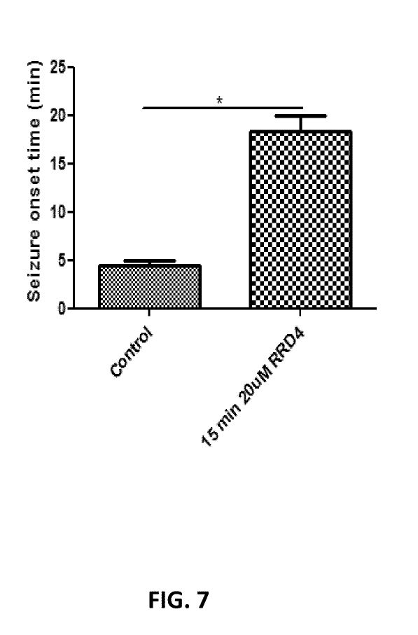

To further examine effects of RRD4 on neural excitability, Applicant used a reliable model of epileptic activity in vitro. Epileptogenic activity was induced using the zero magnesium model. In this model, neural hyperexcitability rises in part due to the opening of the NMDA glutamate receptor and decrease in the membrane surface charge screening. Applicant compared time it takes to form the first seizure (seizure threshold). Brain slices that were pre-incubated in RRD4 had a significant effect in delaying first seizure formation in the zero magnesium solution (FIG. 7). This result suggests that RRD4 in part maybe acting through NMDA receptor, potentially inhibiting its activity and glutamatergic hyperexcitability. Overall, RRD4 pretreatment prolonged the seizure onset time in seizure induced rats (FIG. 7).

RRD4 has a significant effect on neural activity in vitro. Applicant has shown that RRD4 controls action potential formation and abnormal epileptic hyperexcitability. Action potential modulation is likely affecting the two main currents responsible for the action potentials—voltage gated sodium and potassium currents. In epilepsy model of zero magnesium, RRD4 significantly delayed the time to the first seizure formation. This effect on neural hyperexcitability could be mediated by the NMDA glutamate receptors

Without further elaboration, it is believed that one skilled in the art can, using the description herein, utilize the present invention to its fullest extent. The embodiments described herein are to be construed as illustrative and not as constraining the remainder of the disclosure in any way whatsoever. While the preferred embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of treatment or prevention of a neurodegenerative disease comprising administering to a subject in need thereof a therapeutically effective amount of a composition comprising:
one or more isolated monomeric platinum complexes comprising a platinum center selected from Pt(II) and Pt(IV) and having a formula selected from I, II, III and IV: 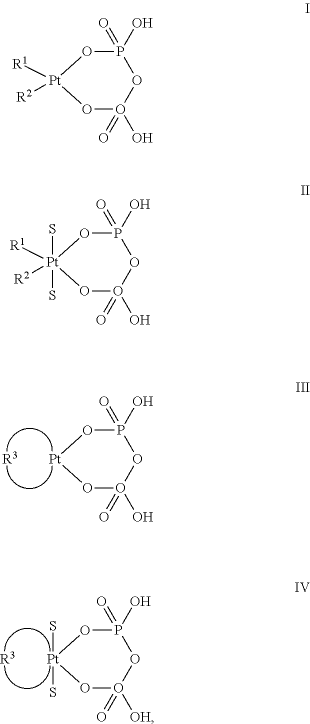
wherein R¹ and R² each is independently selected from substituted or unsubstituted aliphatic or aromatic amines;
wherein when one of R¹ and R² is NH₃, the other of R¹ and R² is not NH₃;
wherein each R³ is selected from substituted or unsubstituted aliphatic or aromatic diamines; and
wherein S is independently selected from hydroxide, acetic acid, butyric acid, and alpha-hydroxy acids; or pharmaceutically acceptable salts thereof.

2. The method of claim 1, wherein the composition is effective in modulating at least one gene selected from the group consisting of NMDA-receptor, ATF, PPT2, HPD, EGR2, SLC7A11, FosB, SQSTM1, TMEM106B, RAB27A, STOX1, and SLC39A3.

3. The method of claim 1, wherein the neurodegenerative disease is selected from the group consisting of amyotrophic lateral sclerosis, Alzheimer's disease, stroke, epilepsy, Parkinsons, Huntington's disease, epilepsy, diabetes associated peripheral neuropathy, leg and foot ulcerations associated with diabetes and pain, and sleep loss induced by diabetes associated neuropathy.

4. The method of claim 1, wherein the administration is intravenously, orally, subcutaneously, intramuscularly, intraocularly or transdermally.

5. The method of claim 1, wherein the one or more isolated monomeric platinum complexes are selected from the group consisting of 1,2-Ethanediamine(dihydrogen pyrophosphato)platinum(II), (trans-1,2-cyclohexanediamine)(dihydrogen pyrophosphato)platinum(II), cis-diammine-trans-dihydroxo(dihydrogen pyrophosphato)platinum(IV), 1,2-Ethanediamine-trans-dihydroxo(dihydrogen pyrophosphato)platinum(IV), and trans-1,2-cyclohexanediamine)-trans-dihydroxo(dihydrogen pyrophosphato)platinum(IV).

6. The method of claim 1, wherein the composition further comprises an adjuvant, a diluent, a vehicle or a pharmaceutically acceptable carrier.

7. A method of modulating expression of at least one gene associated with neuronal cell survival or stability, comprising:
contacting the cell with effective amounts of a composition comprising:
one or more isolated monomeric platinum complexes comprising a platinum center selected from Pt(II) and Pt(IV) and having a formula selected from I, II, III and IV: 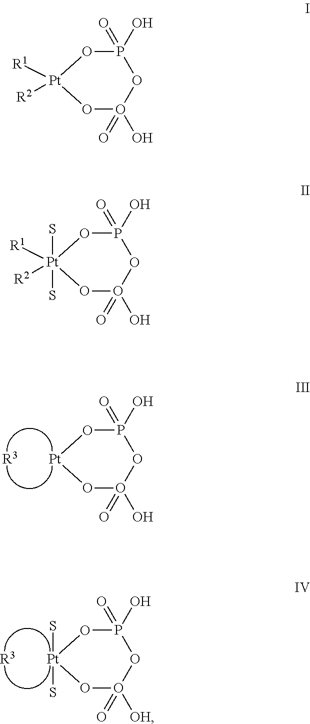
wherein R¹ and R² each is independently selected from substituted or unsubstituted aliphatic or aromatic amines;
wherein when one of R¹ and R² is NH₃, the other of R¹ and R² is not NH₃;
wherein each R³ is selected from substituted or unsubstituted aliphatic or aromatic diamines;
wherein S is independently selected from hydroxide, acetic acid, butyric acid, and alpha-hydroxy acids; or pharmaceutically acceptable salts thereof.

8. The method of claim 7, wherein the at least one gene is selected from the group consisting of NMDA-receptor, ATF, PPT2, HPD, EGR2, SLC7A11, FosB, SQSTM1, TMEM106B, RAB27A, STOX1, and SLC39A3.

9. The method of claim 7, wherein the one or more isolated monomeric platinum complexes are selected from the group consisting of 1,2-Ethanediamine(dihydrogen pyrophosphato)platinum(II), (trans-1,2-cyclohexanediamine)(dihydrogen pyrophosphato)platinum(II), cis-diammine-trans-dihydroxo(dihydrogen pyrophosphato)platinum(IV), 1,2-Ethanediamine-trans-dihydroxo(dihydrogen pyrophosphato)platinum(IV), and trans-1,2-cyclohexanediamine)-trans-dihydroxo(dihydrogen pyrophosphato)platinum(IV).

10. The method of claim 7, wherein the composition further comprises an adjuvant, a diluent, a vehicle or a pharmaceutically acceptable carrier.
